(19) United States
(12) Patent Application Publication
(10) Pub. No.: US 2002/0035241 A1
(43) Pub. Date: Mar. 21, 2002

(54) Process for the separation and/or isolation of plasma proteins by means of annular chromatography

(76) Inventors: Andrea Buchacher, Wien (AT); Djuro Josic, Wien (AT); Gerhard Gruber, Wien (AT)

Correspondence Address: LAW OFFICES OF, JACOBSON HOLMAN, PROFESSIONAL LIMITED LIABILITY COMPANY, 400 SEVENTH STREET, N.W., WASHINGTON, DC, 20004, US

(21) Appl. No.: 09/879,814

(22) Filed: Jun. 13, 2001

Related U.S. Application Data
(63) Continuation of application No. 09/674,530, now abandoned.
Application No. 09/674,530 is a 371 of international application No. PCT/EP99/03659, filed on May 27, 1999.

(30) Foreign Application Priority Data
May 27, 1998 (DE) 198 23 814.2

(51) Int. Cl. C07K 14/745; C07K 1/16

(52) U.S. Cl. 530/380; 530/417

(57) ABSTRACT

A process separates and/or isolates plasma proteins, especially human plasma proteins, from a mixture containing plasma proteins, wherein the mixture is applied to a separation medium having an annular design and having a layer of application medium applied thereon; the separation medium having the annular design is rotated essentially vertically about an axis which is defined in the direction of flow of the mixture through the separation medium having the annular design; an eluent is passed through the separation medium having the annular design; and fractions exiting at the end of the separation medium having the annular design are collected.

This is a continuation of Ser. No. 09/674,530, filed Nov. 24, 2000, which is a 371 of PCT/EP99/03659, filed May 27, 1999, the disclosures are incorporated herein by reference.

The present invention relates to a process for the separation and/or isolation of plasma proteins from a mixture containing plasma proteins.

Plasma proteins play an important role in many physiological processes. Thus, for example, the vitamin K dependent plasma glycoproteins play a prominent part in the blood clotting cascade.

Thus, the recovery of plasma proteins is an important technical process. The starting material for the preparation of plasma proteins is already relatively valuable, because it is obtained, for example, from donated blood. Both for economical and for ethical reasons, it is important to provide processes which yield the desired plasma proteins with high recoveries and in high activities. Today, in addition to recombinant preparation methods, plasma proteins are usually obtained from blood plasma by using conventional chromatographic methods.

It has been found that high molecular weight substances can be separated and isolated within an order of magnitude by means of annular chromatography. It has further been found that human plasma proteins can be obtained in purified form in a surprisingly simple way even from complex mixtures. This is achieved by a process comprising the following steps: the mixture containing the plasma proteins, especially human plasma proteins, is applied to a separation medium having an annular design. The separation medium having the annular design is rotated vertically about an axis which is defined in the direction of flow of the mixture through the separation medium having the annular design. An eluent is passed through the separation medium having the annular design, and fractions exiting at the end of the separation medium having the annular design are collected.

The use of annular chromatography for the desalting of mixtures containing bovine serum albumin has already been described by K. Reissner et al., Journal of Chromatography A, 763 (1997), 49 to 56. Reissner et al. held the opinion that the enormous difference in size between the substances to be separated, namely bovine serum albumin and low molecular weight salts, was critical to the chromatographical purification's being successful. Bloomingburg et al. (Ind. Eng. Chem. Res. 30, 1061-1067 (1991) have shown that a mixture of the pure components, bovine serum albumin and bovine hemoglobin, can be separated by means of a cation exchanger having an annular design. Elution is carried out with a single eluent under isocratic conditions. A number of application means are employed for applying sample material.

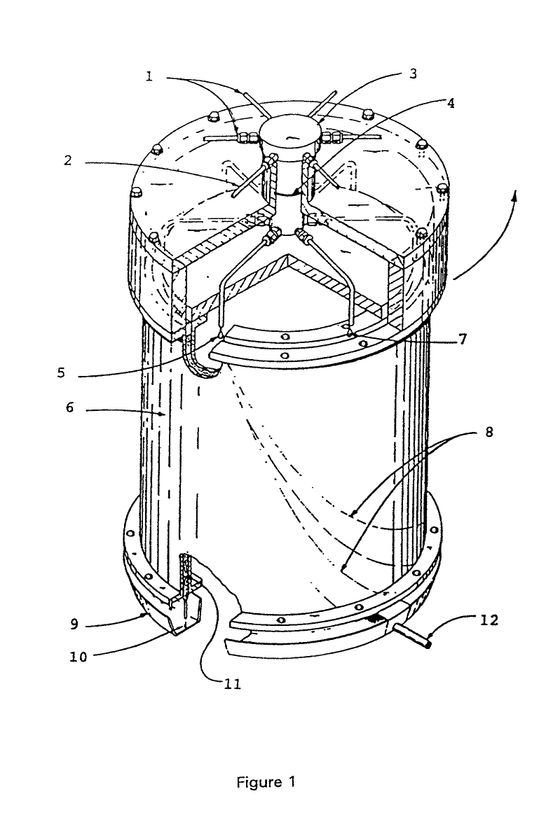

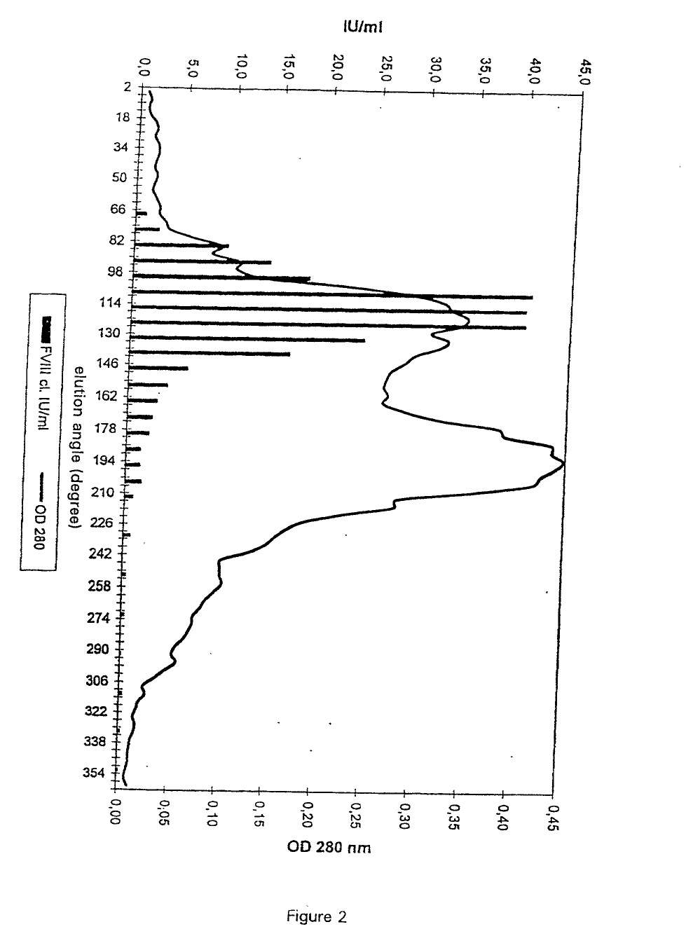

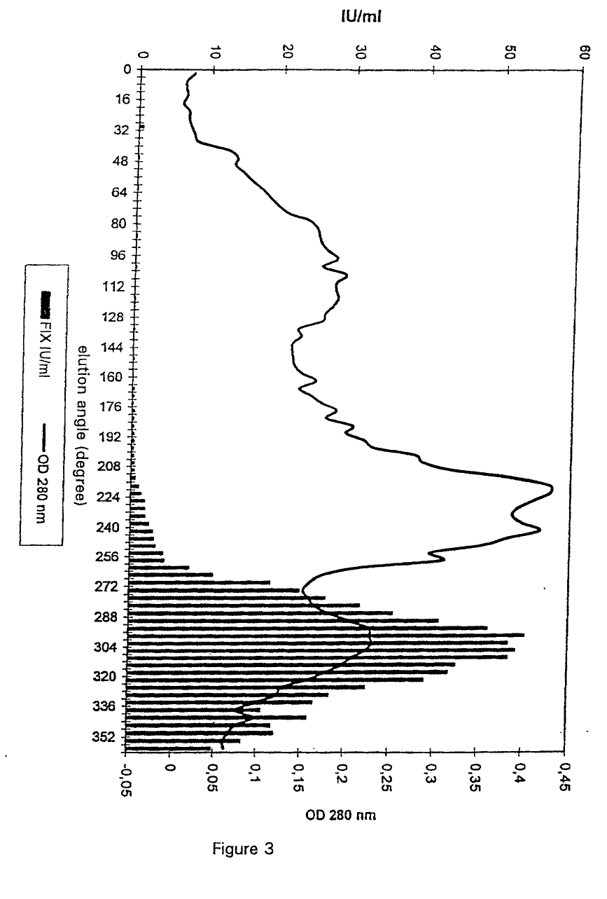

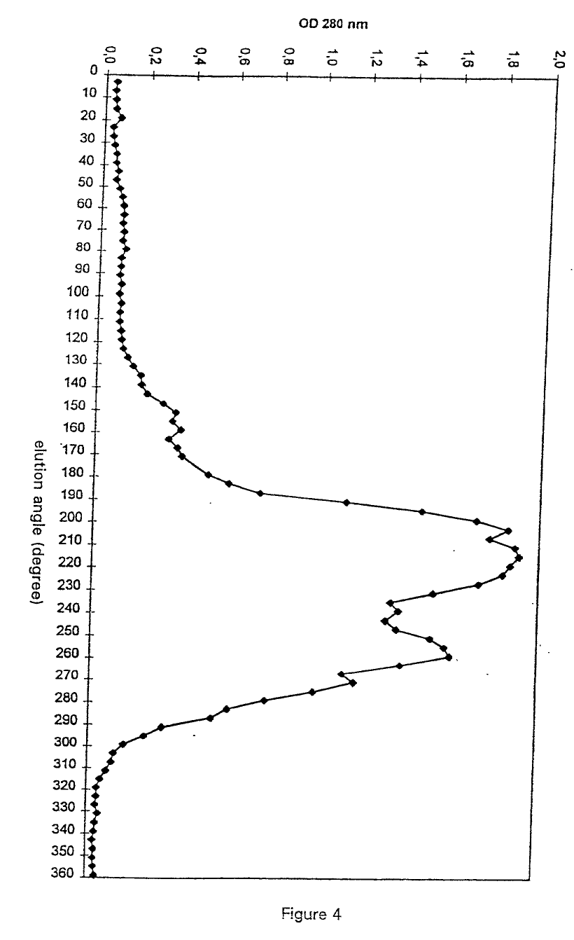

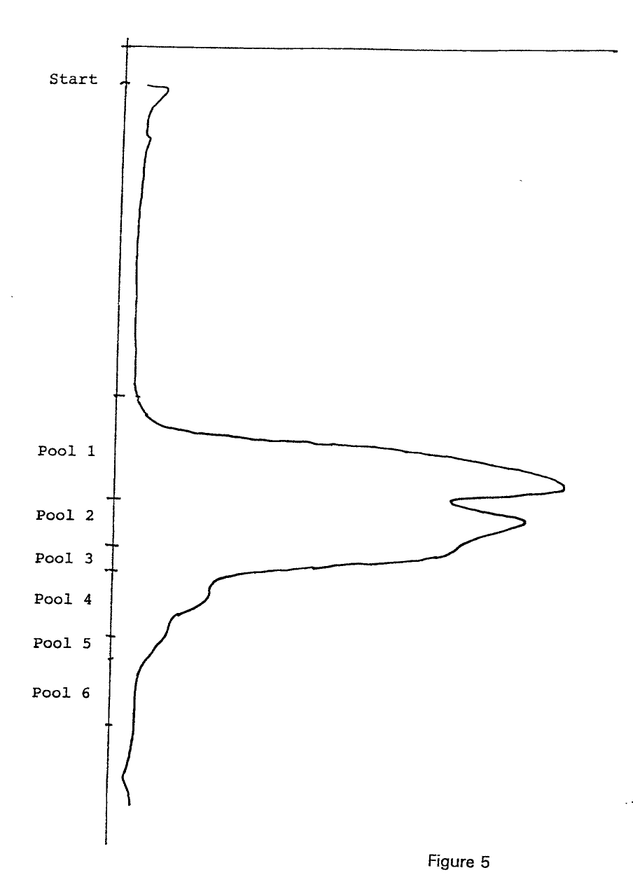

For performing the process according to the invention, it is preferred to use the device described in the publication mentioned. FIG. 1 schematically shows the subject device. FIG. 2 shows a typical chromatogram of a complex of factor VIII and von Willebrand factor, and FIG. 3 shows a corresponding chromatogram of factor IX. FIG. 4 shows a separation of BSA and IgG. FIG. 5 shows the result of a conventional separation by column chromatography.

Preferably, blood plasma or mixtures containing virus-inactivated plasma proteins are used as the source of the plasma glycoproteins to be separated and/or isolated. Said mixture containing plasma proteins is preliminarily processed, e.g., in the usual way. For virus inactivation, in particular, methods are employed which are known as solvent/detergent methods. Thus, such a method is described, in particular, in B. Horowitz et al., “Blood” 79 (1992), 826 to 831.

Therefore, the mixture may also contain a detergent which may also, in addition, suppress undesirable non-specific interactions of the plasma proteins with the separation media. The mixture employed can be obtained not only from blood plasma, but it may also be provided as a fraction of a cell culture containing the human plasma proteins which have been prepared by genetic engineering.

The separation medium having the annular design preferably consists of materials used for adsorption chromatography, such as ion-exchange, gel permeation, molecular size exclusion or affinity chromatography or chromatography based on hydrophobic interactions. The usual materials are employed.

It may also be preferred to use more than one separation medium in one chromatographic configuration. Thus, a chromatographic column may be packed with layers of different separation media to achieve a combined separation effect. Not only similar media can be selected, but also very different ones, e.g., an anion exchanger and a medium for hydrophobic interaction chromatography, or two anion exchangers of different strength, or an adsorption material and a material for gel permeation chromatography. However, care should be taken that an undesirable mixing of the materials does not occur and that the buffers used are suitable for all the separation materials employed.

A preferred separation medium is employed as a compact block material (monolith) which already has an annular shape suitable, e.g., for the separation column. This medium can be combined, for example, with a loose packing of a chromatographic material. The block-shaped media suitable as separation media are not only inorganic or organic monoliths obtained by block polymerization which are disclosed, e.g., in EP-A-0 320 023, incorporated herein by reference. Support materials such as shaped membranes or membranes having a textile structure, such as based on cellulose, can also be employed for annular chromatography. The monoliths or support materials are optionally surface-modified to obtain ligands which may be required.

Another advantageous embodiment relates to the favorable application of the sample material to the separation medium. It has been found that an application medium for the well-aimed local application of the sample material should preferably be used. Any material can be employed which serves to shortly localize the sample and which is not mixed with the separation medium. This can be ensured by specifically selecting the material, in which selection the density and the condition of the surface of the material play a role. However, it is also possible to use a device which provides a separation layer between the application medium and the separation medium. Spherical particles, e.g., glass beads, are preferably employed as application media, the particles optionally being treated to prevent nonspecific interactions.

It may be preferred to provide the spherical particles with a hydrophobic surface, if they do not initially have a sufficiently hydrophobic surface. For example, glass particles hydrophobized with reagents such as silanization reagents can be used. The particle size of the glass particles is preferably in a range of from 20 to 500 μm. The spherical particles cover the separation medium having the annular design and protect it from mechanical impacts brought about by the application device during the rotation of the device holding the separation medium having the annular design. If surfaces are employed which are already relatively hydrophobic, such as those provided, for example, by appropriate plastic particles, it is not necessary to hydrophobize their surfaces.

The plastics of which the spherical particles consist may be, in particular, polymethacrylates and polystyrene/divinylbenzene.

The plasma proteins obtainable by the process according to the invention include, in particular, inter-α-trypsin inhibitor, immune globulins, such as IgG, human serum albumin or glycoproteins from the clotting cascade. Preferably, vitamin K dependent factors of the blood clotting cascade, such as factor IX, other blood clotting factors, such as factors VIII, XI and XIII, antithrombin III, α₁-antitrypsin and thrombin are obtained from a plasma fraction. For example, factor VIII is separated from accompanying proteins, such as fibrinogen, in a fraction of the cryoprecipitate by anion exchange chromatography, optionally combined with molecular size exclusion chromatography. Factor IX in admixture with vitamin K dependent plasma proteins is also employed as a starting material, wherein the accompanying plasma proteins may be effectively separated by means of a combination of anion-exchange and affinity chromatography or molecular size exclusion chromatography, but also by means of hydrophobic interaction chromatography.

One aspect of the present invention is the use of human proteins which have themselves to be purified with great care from a complex mixture of proteins. It is to be noted that the human proteins, in particular, must be activated or denaturated to as low an extent as possible, and the mixture of human plasma proteins is difficult to separate due to the similarity of the physico-chemical properties between the human plasma proteins. It has proven advantageous that the process according to the invention can above all be employed for separating at least two different human proteins without substantially affecting their biological activity.

Due to the complexity of the starting materials, it is also often necessary to apply and separate a large quantity of proteins. A continuous procedure is highly advantageous since the capacity of the column can be used in a virtually unlimited way. With the annular design, a really continuous procedure can be provided for the first time which enables not only the application of the sample, the separation of the plasma proteins and the fractioning to be performed simultaneously. The separation medium can even be simultaneously regenerated and equilibrated. While one region of the separation medium is regenerated, another region can be equilibrated with a buffer, which is then continuously employed for applying the sample. Thus, the chromatographic plant can be used on a long-term basis for days and weeks. The effective capacity of the column can be increased thereby, depending on the duration of the continuous chromatographic process. This is advantageous, above all, for gel permeation and molecular size exclusion chromatographies, since these types of chromatography are limited for the separation of proteins by their relatively low capacities according to the prior art. This truly continuous procedure is also fundamentally distinct from other quasi-continuous column-chromatographic methods in which a series of physically separated compartments with separation media are employed (“simulating moving bed”).

The rotational speed is preferably selected within a range of from 100 to 2000 degrees per hour, preferably from 600 to 2000 degrees per hour. It has been established that at the higher speeds, the fractioning is advantageous, and the residence time of the human proteins in the separation medium can be minimized. Usually, flow rates within a range of from 1 to 2000 cm/min, preferably from 15 to 300 cm/min, are selected for the separation.

The process according to the invention is suitable, above all, for use on an industrial scale for the preparative separation of human plasma proteins. Thus, depending on the specific activity of the proteins, recoveries of from at least 40 to almost 100% can be obtained. If prepurified proteins are used as the starting material, a recovery of at least 90%, preferably at least 95%, may even be achieved in the further purification, as “polishing”. Thus, pharmaceutical preparations containing plasma proteins, especially human plasma proteins, in a highly purified form can be provided in an economical way.

In addition, annular chromatography has the advantage that it can be performed continuously.

The separation of human plasma proteins is achieved particularly well by adsorption chromatography using a step gradient, using at least two different elution solutions of different eluting strength. It has also proven advantageous to provide only one device for applying the sample material lest the separation medium should be overloaded by the sample material having the high protein concentration.

For monitoring the fractioning process and for purposefully selecting the fractions to be collected, a monitor is advantageously used which continuously monitors the protein concentration in the eluates. As said monitor, a suitable detector, for example, a photometer, may be employed.

FIG. 1 schematically shows a preferred device for performing the process according to the invention. The support material having an annular design is provided between two concentric cylinders. The outer cylinder is closed with a flange at the top end. Preferably, the cylinder itself is made of a steel material. The inner cylinder is preferably made of a durable material, and shorter than the outer one, so that a space results at the top end which allows to distribute the eluent uniformly on the whole separation medium having the annular design. At the head of the chromatographic unit, the sample application device and the eluent application device are provided. At the bottom of the unit, both cylinders are connected with a second flange. This flange contains a number of equidistantly spaced holes, the holes being preferably distributed uniformly in intervals of angles from at least 2° to 180°, preferably from 4 to 36°. Preferably, flexible capillaries are inserted in these holes which capillaries lead to a fraction collector, for example.

In detail, FIG. 1 shows an eluent inlet 1, a sample inlet 2, a stationary inlet distributor 3, an inlet pressure closure 4, a continuous sample flow 5, a rotating annular chromatography unit 6, an eluent flow 7, a separated sample 8, a stationary collector means for the waste eluent 9, an eluent outlet 10, a support material having an annular design 11, and a product outlet 12.

FIG. 2 shows an annular chromatogram of factor VIII/von Willebrand factor.

FIG. 3 shows an annular chromatogram of factor IX.

FIG. 4 shows an annular chromatogram of immune globulin G and bovine serum albumin (BSA), wherein immune globulin G elutes first.

FIG. 5 shows a size exclusion chromatogram of a factor VIII concentrate on a conventional discontinuous axial column for comparison.

The invention is further illustrated by means of the following Examples.

### EXAMPLE 1

An annular chromatography device according to Journal of Chromatography A, 563 (1997), 49 to 56, was packed with 2 l of Fractogel BioSec EMD 650 (S). The duration of the application of the mixture to be separated depends on the elution flow rate. After passing the bottom of the support medium having the annular design, the sample application was continued for another hour to achieve equilibrium. Thereafter, the collection of fractions was begun. The apparatus used in the Examples had exits at the bottom of the chromatographic unit at a distance of 2° each. The outlet holes were respectively combined in pairs to give one fraction.

Separation of human polyclonal IgG and BSA

Mixture to be separated: 2.5 mg/ml of IgG and 5 mg/ml of BSA in aqua injectabilia.

Buffer:

27.5 mM disodium hydrogenphosphate dihydrate

12.5 mM sodium dihydrogenphosphate dihydrate

0.2 mM sodium chloride

pH=7.2

The result of the separation is shown in FIG. 4.

### EXAMPLE 2

Separation of factor IX concentrate

Sample solution: Factor IX lyophilizates with 500 IU (Octanyne, Octapharma GmbH) were dissolved and adjusted to a concentration of from 20 to 500 IU/ml.

Buffer:

20 mM sodium citrate

0.2 M sodium chloride

2 mM calcium chloride

pH=7.4

The chromatogram of the separation is shown in FIG. 3.

### EXAMPLE 3

Separation of factor VIII concentrate

Mixture to be separated: Factor VIII lyophilizate with 500 IU (Emoclot) was dissolved and adjusted to a concentration of from 20 to 500 IU/ml.

Buffer:

20 mM sodium citrate

0.2 M sodium chloride

2 mM calcium chloride

pH=7.4

The chromatogram of the separation is shown in FIG. 2.

1. A process for the separation and/or isolation of plasma proteins, especially human plasma proteins, from a mixture containing plasma proteins, wherein
said mixture is applied to a separation medium having an annular design and having a layer of application medium applied thereon;
said separation medium having the annular design is rotated essentially vertically about an axis which is defined in the direction of flow of the mixture through the separation medium having the annular design;
an eluent is passed through the separation medium having the annular design; and
fractions exiting at the end of the separation medium having the annular design are collected.

2. The process according to claim 1, characterized in that said application medium comprises spherical particles.

3. The process according to claim 1 or 2, characterized in that said application medium has a treated surface which prevents non-specific interactions with components to be separated from the mixture.

4. The process according to claims 1 to 3, wherein said mixture is blood plasma or mixtures containing virus-inactivated plasma proteins.

5. The process according to claims 1 to 4, characterized in that said mixture contains at least two plasma proteins to be separated.

6. The process according to claims 1 to 5, wherein said separation medium having the annular design is used for ion-exchange, gel permeation, molecular size exclusion or affinity chromatography or chromatography based on hydrophobic interactions.

7. The process according to at least one of claims 1 to 6, wherein said plasma proteins are inter-α-trypsin inhibitor, α₁-antitrypsin, antithrombin III, immune globulins, such as IgG, human serum albumin or glycoproteins, preferably from the clotting cascade, or vitamin K dependent blood clotting factors.

8. The process according to at least one of claims 1 to 6, characterized in that said plasma proteins are selected from blood clotting factors VIII, IX, and thrombin.

9. The process according to any of claims 1 to 8, characterized in that the functions of mixing, of separating the plasma proteins and of the fractioning are performed continuously.

10 The process according to any of claims 1 to 9, characterized in that the separation medium is continuously regenerated and equilibrated, simultaneously with the separation of the plasma proteins.

11. The process according to any of claims 1 to 10, characterized in that when a material for adsorption chromatography is used as the separation medium, at least two different eluents are simultaneously passed through said separation medium having the annular design.

12. The process according to any of claims 1 to 11, characterized in that at least two different separation media are employed in layers.

13. The process according to any of claims 1 to 12, characterized in that a polymeric block material is employed as said separation medium.
